(19) United States
(12) Patent Application Publication
(10) Pub. No.: US 2003/0163176 A1
(43) Pub. Date: Aug. 28, 2003

(54) Personal thermotherapeutic apparatus having chiropractic function

(76) Inventor: Kyung-Han Bae, Kyungnam (KR)

Correspondence Address: Notaro & Michalos, 350 Fifth Avenue, Suite 6902, Empire State Building, New York, NY, 10118-0110, US

(21) Appl. No.: 10/110,951

(22) PCT Filed: Oct. 18, 2001
(86) PCT No.: PCT/KR01/01749

(51) Int. Cl. A61F 7/00

(52) U.S. Cl. 607/96

(57) ABSTRACT

Disclosed is a personal thermo-therapeutic apparatus having an acupressure function. The thermo-therapeutic apparatus is adapted to be connected to a typical thermo-therapeutic and low frequency generating apparatus. The personal thermo-therapeutic apparatus comprises a body including at least one acupressure projection having a low frequency oscillatory member, and an electro-thermal wire arranged in the body. The body and the acupressure projection are made of silicon material, and the body includes a wearing belt.

### TECHNICAL FIELD

The present invention relates to a personal thermo-therapeutic apparatus having low frequency therapy, thermo-therapy, and acupressure function.

### BACKGROUND ART

Generally, a low frequency therapy apparatus is to treat a subject by stimulating a nerve or muscle with an electrical signal consisting of a low frequency. In particular, since such an apparatus has an advantage that the treatment such as neurolepsis, stitch, myalgia or the like can be performed by himself or herself, the apparatus is widely used.

Recently, since a new low frequency therapy apparatus employing a microprocessor for adjusting frequency, time and intensity is available, the selection range of the subject may be further extended. The prior low frequency therapy apparatus is limited to the kneading, massaging and acupressure effects, and no hot cataplasm is provided, thereby deteriorating the remedial value.

In order to solve the drawback, a thermo-therapeutic apparatus having an acupressure and/or thermo-therapeutic function, and a low frequency therapy apparatus having a low frequency and/or thermo-therapeutic function are proposed. Accordingly, the subject has to use the above two apparatus to achieve the low frequency therapy, thermo-therapy, and acupressure function. In addition, since the prior apparatus is made of plastic, ceramic, or stone material, if it is ruptured, it is injurious to human body, and induces an illness. Furthermore, the low frequency therapy apparatus consists of a low frequency therapy body which is separated from a belt, thereby inconveniencing the subject.

### DISCLOSURE OF THE INVENTION

Therefore, an object of the present invention is to solve the problems involved in the prior art and to provide a personal thermo-therapeutic apparatus employing a low frequency therapy, a thermo-therapy, and an acupressure function and having functionality, convenience, and stability by integrally forming a body with a wearing belt.

In order to accomplish the above mentioned object, according to one aspect of the present invention, there is provided a personal thermo-therapeutic apparatus having an acupressure function, the thermo-therapeutic apparatus being connected to a thermo-therapeutic and low frequency generating apparatus, the personal thermo-therapeutic apparatus comprising: a body including at least one acupressure projection having a low frequency oscillatory member; and an electro-thermal wire arranged in the body. The body and the acupressure projection are made of silicon material, and the body includes a wearing belt.

### BRIEF DESCRIPTION OF THE DRAWINGS

The above objects, other features and advantages of the present invention will become more apparent by describing the preferred embodiment thereof with reference to the accompanying drawings, in which:

FIG. 1 is a perspective view illustrating the construction of a personal thermo-therapeutic apparatus having an acupressure function according to one preferred embodiment of the present invention.

FIGS. 2 and 3 are top views illustrating a body and cover of a personal thermo-therapeutic apparatus according to present invention.

FIG. 4 is an exploded view of a portion of a personal thermo-therapeutic apparatus according to present invention.

### BEST MODE FOR CARRYING OUT THE INVENTION

Now, a preferred embodiment of the present invention will be described in detail with reference to accompanying drawings wherein like reference numerals designate corresponding parts throughout several views. In addition, the detained description on the function or construction of the known parts will be omitted for clarity.

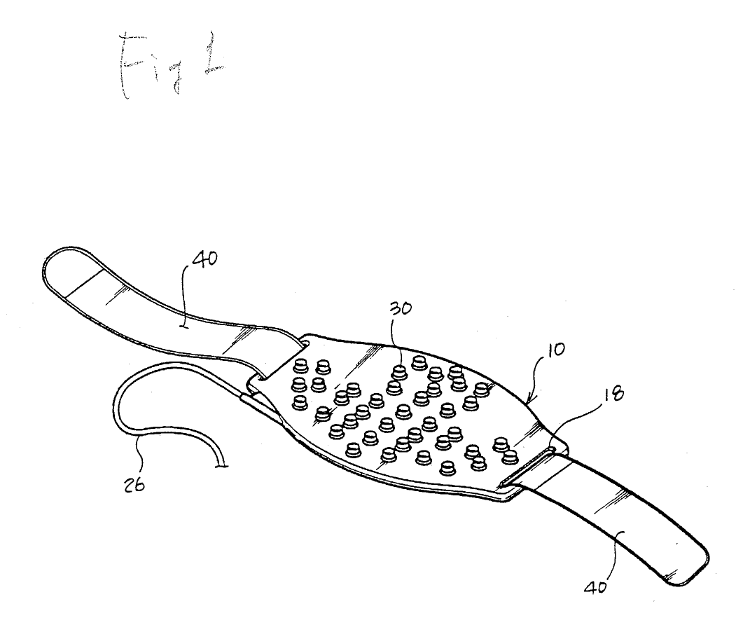

FIG. 1 is a perspective view illustrating the construction of a personal thermo-therapeutic apparatus having an acupressure function according to one preferred embodiment of the present invention. Referring to FIG. 1, the personal thermo-therapeutic apparatus of the present invention comprises a body 10, the body including at least one acupressure projection 30 having a low frequency oscillatory member 32. A heating element, i.e., electro-thermal wire 24 is arranged in the body, and the body also includes a wearing belt 40. The wearing belt 40 is coupled to latching slot formed both sides of the body 10, so that the thermo-therapeutic apparatus may be conveniently worn on the body of the subject.

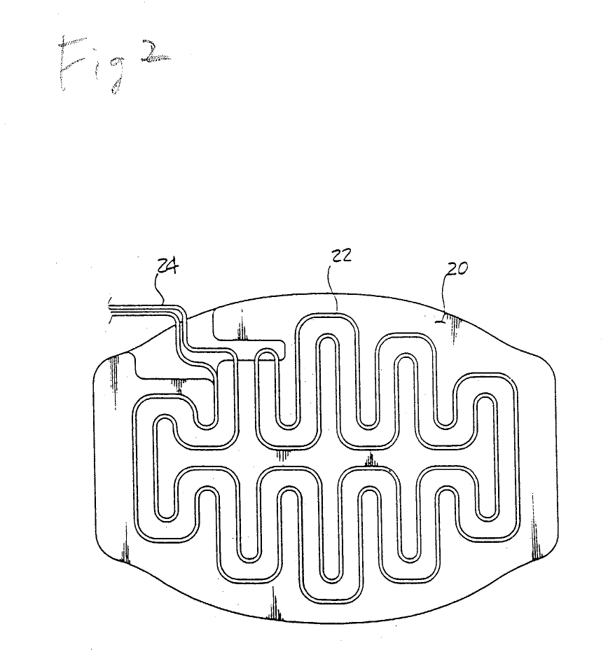

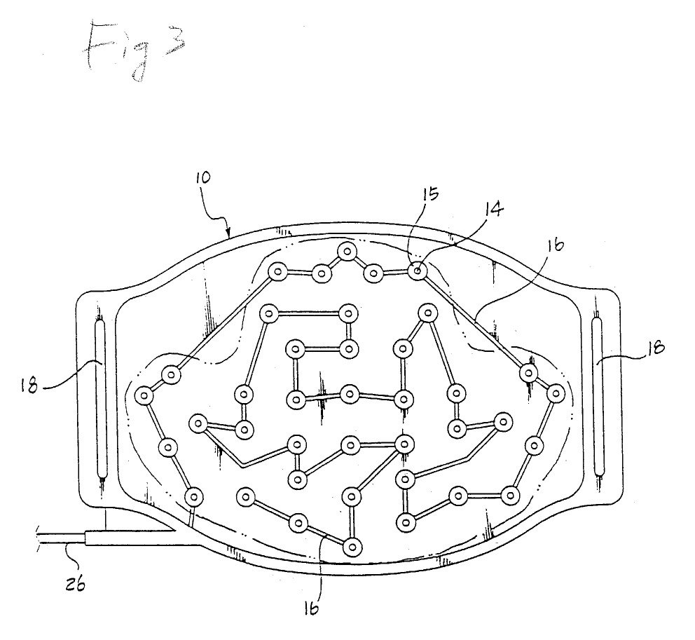

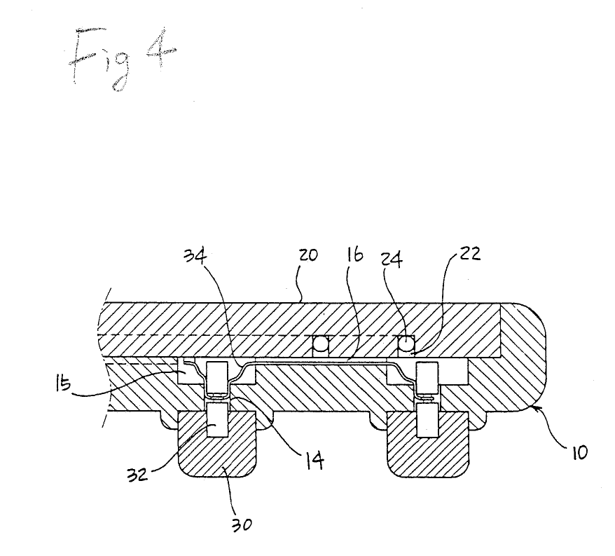

FIGS. 2 and 3 are top views illustrating a body and cover of a personal thermo-therapeutic apparatus according to present invention, and FIG. 4 is an exploded view of a portion of a personal thermo-therapeutic apparatus according to present invention. Referring to FIGS. 2 to 4, the body 10 includes a hollow portion 15 for receiving the low frequency oscillatory member 32, the hollow portion communicating with a passage 16 for receiving an electric wire 34. The body is provided with a groove 22 for receiving the electro-thermal wire 24. A cover plate 20 is engaged to a recess portion 12 formed in the body 10. The hollow portion 15 is formed in the recess portion 12, and a hole 14 is formed in a center of the hollow portion 15. The low frequency oscillatory member 32 is inserted into the hole 14, and the electric wire is run along the passage 16. A lower portion of the oscillatory member 32 is mounted onto the acupressure projection 30 coupled to the body 10.

A tortuous groove 22 is formed in the entire surface of the cover plate 20, and the electro-thermal wire 24 is inserted into the tortuous groove 22. One example of the arrangement of the electro-thermal wire 24 is shown in FIG. 2, and the arrangement may be altered, if necessary. In addition, the oscillatory member 32 and the acupressure projection 30 are arranged corresponding to spots on the body of the subject suitable for acupuncture. Preferably, the oscillatory member 32 and the acupressure projection 30 are arranged to cover the abdominal region of the subject on the whole, as shown in FIG. 3, with they positioned corresponding to the spots. The arrangement may be altered depending upon a lesion to be treated. Furthermore, the thermo-therapeutic apparatus according to present invention can be easily attached to a meridian portion or a meridian pathway portion such as back, waist, abdominal region, hand, foot, hips, breast or the like.

The body 10 and the acupressure projection 30 are made of silicon material harmless to the human body, thereby protecting the subject from being diseased. In addition, a cable 26 accommodating both the electric wire 34 and the electro-thermal wire 24 is connected to a typical thermo-therapeutic and low frequency generating apparatus.

### INDUSTRIAL APPLICABILITY

As apparent from the above description, the personal thermo-therapeutic apparatus according to the present invention employs a low frequency therapy, a thermo-therapy, and an acupressure function. The body and the acupressure projection are made of silicon material harmless to the human body, so that the apparatus can be directly contacted with the skin of the subject. Since the wearing belt is integrally with the body, the subject can conveniently wear the apparatus.

1. A personal thermo-therapeutic apparatus having an acupressure function, the thermo-therapeutic apparatus being connected to a thermo-therapeutic and low frequency generating apparatus, the personal thermo-therapeutic apparatus comprising:
a body 10 including at least one acupressure projection 30 having a low frequency oscillatory member 32; and
an electro-thermal wire 24 arranged in the body;
the body 10 and the acupressure projection 30 made of silicon material.

2. The personal thermo-therapeutic apparatus as claimed in claim 1, wherein the body includes a wearing belt.

3. The personal thermo-therapeutic apparatus as claimed in claim 1 or 2, wherein the acupressure projections 30 are arranged corresponding to spots on a human body suitable for acupuncture.

4. The personal thermo-therapeutic apparatus as claimed in claim 1 or 2, wherein the body 10 includes a hollow portion 15 for receiving the low frequency oscillatory member 32, the hollow portion communicates with a passage 16 for receiving an electric wire 34, and the body is provided with a groove 22 for receiving the electro-thermal wire 24.
